(19) United States
(12) Patent Application Publication
(10) Pub. No.: US 2005/0136082 A1
(43) Pub. Date: Jun. 23, 2005

(54) Polymeric odor absorption ingredients for personal care products

(75) Inventors: David S. Soane, Chestnut Hill, MA (US); Kyle L. Fujdala, San Francisco, CA (US)

Correspondence Address: JACQUELINE S LARSON, P O BOX 2426, SANTA CLARA, CA, 95055-2426, US

(73) Assignee: Cosmetica, Inc.

(21) Appl. No.: 10/966,365

(22) Filed: Oct. 15, 2004

Related U.S. Application Data
(63) Continuation of application No. PCT/US03/11669, filed on Apr. 16, 2003.
(60) Provisional application No. 60/373,374, filed on Apr. 16, 2002.

(51) Int. Cl. A61K 6/00

(52) U.S. Cl. 424/401

(57) ABSTRACT

The present invention is directed to modified polyamines and to compositions comprising one or more modified polyamines as the main odor absorbing ingredient(s) for use in odor absorbing or deodorizing personal care products. The invention is further directed to personal care products with deodorizing properties using the disclosed compositions and to methods of making and using the compositions and products.

The present invention is a continuation of co-pending International Patent Appln. No. PCT/US03/11669, filed Apr. 16, 2003 and designating the United States of America, which application claims the benefit of Provisional U.S. patent application Ser. No. 60/373,374, filed Apr. 16, 2002; the entire disclosures of both of which are incorporated herein by reference.

### FIELD OF THE INVENTION

The present invention is directed to the field of personal care products with odor absorbing and/or odor enhancing properties.

### BACKGROUND OF THE INVENTION

The elimination or significant reduction of malodorous axillary (body) odors (including foot odor) is a highly desirable attribute for personal care products (creams, powders, gels, lotions, sprays, patches, etc). Hence, odor absorption has long been an area of active research and development for the personal care industry, resulting in numerous compositions that exhibit varying degrees of effectiveness on the human skin. Although the ingredients of such commercial compositions are similar (e.g., deodorizing and/or antiperspirant active(s), suspending or thickening agents, fragrance, a suitable solvent, etc.), these can be used in many forms: solid emulsion sticks, suspensoid sticks, roll-on liquids, aerosol and non-aerosol sprays, creams, lotions, powders, etc. Of the various forms available, underarm deodorant products tend to utilize stick or roll-on forms, foot odor control products are usually in powdered or spray form, and various deodorizing soaps (hand and body) are in cream or lotion form. Despite their popularity, emulsion sticks (containing an emulsified solution of the active deodorizing ingredients) tend to be tacky, prone to deposition of a visible residue, and unstable, resulting in non-uniform behavior. Additionally, suspensoid sticks (with the active ingredients suspended throughout the stick in powdered form with no solvent) have a tendency to leave an unpleasant powdered residue behind. Foot odor reducing products are best formulated as powders to be used in the shoes. Odor absorbers to be used on the entire body are best suited in cream, lotion, or spray form.

Many of the chemical components of axillary odor are the waste by-products of certain bacteria that live off of the secretions from human sweat glands. These species of bacteria are called lipophilic diptheroids. At least three-dozen molecules with potentially offensive odors have been identified in body odor (Preti, G. et al, J. Chem. Ecology, 1991, 17, 1469; Preti, G. et al, J. Chem. Ecology, 1992, 18, 1039; Preti, G. et al, J Chem. Ecology, 1996, 22, 237; Proc. Nat. Acad. Sci. USA, 1996, 93, 6626). Most of them are organic acids, and the main contributor to the odor has been identified as trans-3-methyl-2-hexenoic acid. The chemical components of foot odor have similar origin; they are waste products of the bacteria Brevidium epidermis. Many of these molecules are also organic acids, and the most significant component is isovaleric acid (Kanda, F. et al, Brit. J. of Dermatology, 1990, 122, 771). Fatty acids and some non-acid organic molecules (amines, alcohols, thiols, aldehydes, ketones, phenols, etc.) are also known to contribute to body odor in some instances, although with reduced significance.

Two primary mechanisms have been pursued as a means to counter foul odors emanating from the human skin. The first involves bacteriocidal or bacteriostatic agents that control the proliferation of organisms naturally found on the skin. However, significant concerns exist over extended use of such bioactive agents, since all organisms are known to mutate so as to create agent-resistant strains over time. Hence, use of such agents in every-day deodorant products is generally not recommended.

The second mechanism to counter unpleasant body odor relies on physical and/or chemical sequestering of the offensive odor molecules using active ingredients. These active ingredients are capable of strong interactions with odor molecules and they can be further categorized into two broad classes—inorganic and organic components. Inorganic odor absorbing components are considered to be an acceptable option due to their excellent performance and inexpensive cost. Examples of inorganic odor absorbing components that have enjoyed widespread use in personal care products include aluminum, zinc, and zirconium salts. Also, alkali metal (sodium, potassium, etc.) bicarbonate salts, and various metal oxides and hydroxides (such as magnesium hydroxide) are commonly used in odor absorbing products (for personal care and household use). All of these well established ingredients tend to leave a white residue when applied to the human skin, thus detracting from their general appeal. Additionally, many of these cause skin irritation when used in large quantities, therefore their use should be (ideally) on a limited basis. Zeolites (microporous aluminosilicate inorganic materials) are a further oxide material capable of trapping certain organic molecules, however they tend to be less effective when wet (i.e., upon perspiration) and also leave an unpleasant residue. Activated charcoal is another well-known odor absorbing ingredient, but its dark color is detrimental to efforts to formulate a visually pleasing composition (i.e., a colorless product). A further disadvantage of many of these odor absorbing components is that they can be somewhat abrasive (causing a harsh feel) when used in large quantities.

In the arena of organic actives for odor abatement, cyclodextrins and related derivatives have been proposed. Additionally, various functional polymers have been put forward as effective odor absorbing components. Procter and Gamble recently developed cyclodextrins as an effective key ingredient for odor absorption (see U.S. Pat. No. 6,344,218, U.S. Pat. No. 5,911,976, U.S. Pat. No. 5,879,666 and U.S. Pat. No. 5,874,070). However, cylcodextrins are expensive and require significant antimicrobial preservatives to ensure adequate shelf and service life of the formulated products, therefore their use as the primary odor absorption ingredient is impractical in many instances.

Advanced deodorizing preparations containing cationic biopolymers alongside aluminum hydrochlorate and esterase inhibitors (Henkel, U.S. Pat. No. 5,968,488) are cited as being an effective example of the use of functional polymers. The intended function of the biopolymer (preferably chitosan-related polymers) is the suppression of esterase-producing bacteria. An earlier patent by Dainippon Ink (U.S. Pat. No. 4,909,986) provides an extensive list of functional groups that exhibit some odor absorption properties, specifically: (a) ammonium salts of carboxylic acids, ammonium/alkali mixed salts of carboxylic acids, and alkanolamine salts of carboxylic acids; (b) sulfoalkyl groups, sulfonic acids, phosphoric acids and their alkali metal salts, ammonium salts, alkanolamine mixed salts; (c) cationic groups, including quaternized ammonium groups. Polymers containing at least one of these functional groups were cited as needing to have a number average molecular weight of at least 1,000,000. It is well known to those skilled in the art of polymer science and technology that it is difficult for mixtures of different polymers to form thermodynamically compatible systems. This is especially the case when the polymer molecular weights are high. Hence, formulations having more than one such polymeric component tend to be unstable. In addition, interactions among the various functional groups may cause the components to precipitate, thus disrupting the desired homogeneity of the product and causing further instability. Therefore, with organic polymers for odor absorption, it is desirable to choose one polymer with a single type of functional group to be the major (and perhaps the only) active component to facilitate subsequent formulation efforts. Among cationic group-containing polymers for use as odor-absorbing ingredients, U.S. Pat. No. 4,909,986 disclosed poly(dimethylaminoethyl acrylate), copoly(dimethylaminoethyl acrylate/acrylamide), copoly(vinylbenzyltrimethyl ammonium chloride/acrylamide) and copoly(acrylamide/dimethylaminoethyl methacrylate) with high molecular weights (>1,000,000). Unfortunately, these polymers contain hydrolyzable linkages which are particularly vulnerable in the presence of esterase-secreting bacteria.

U.S. Pat. No. 4,244,059, issued to Pflaumer (Proctor and Gamble), taught the use of a water-soluble amine-containing polymer, Tydex-12 (Dow Chemical Co.), as an “odor absorbent compound” for application to fabrics. Tydex-12 was applied to a soft, air-permeable fabric composed of cellulosic fibers, and the treated fabric was then used in the manufacture of odor absorbent underwear (panty-type) garments. The patent made no claims as to the durability of the polymer treatments nor did it suggest any modifications to the polymer to enhance its biocompatibility, reduce its skin irritation sensitivity, and further enhance its properties.

U.S. Pat. No. 5,863,525, issued to Church & Dwight Co, disclosed the use of polyalkylenimines (one type of polyamine) as clarifying agents in order to reduce skin discoloration and/or whitening in odor absorbing personal care products. These polymers (in an unmodified state) were used in quantities from 1.25 to 8% (by weight). No odor absorbing properties of these polymers were claimed in this disclosure. Polyalkylenimines have also been proposed for use in various dental care formulations to act as complexing agents for solubilizing zinc compounds and other inorganic salts that have low-moderate water solubility (U.S. Pat. Nos. 4,522,806 and 4,082,841).

### SUMMARY OF THE INVENTION

The present invention is directed to modified polyamines and to odor absorbing compositions comprising one or more of the modified polyamines as the main odor absorbing ingredient(s) for use in odor absorbing or deodorizing personal care products. The invention is further directed to personal care products with odor-absorbing or deodorizing properties using the disclosed compositions and to methods of making and using the modified polyamines, compositions and products. By “main” or “major” odor absorbing ingredient or component is meant that the odor absorbing ingredient(s) component in the compositions of the invention will be made up of greater than 50% and up to 100% of modified polyamines.

The modifications are proposed to enhance odor absorbing performance of the polyamines, to render the polymers acceptable for use in biological systems, and to add other desirable properties. Examples of other desirable properties provided by the modifications include, but are not limited to: improved gas permeability, elimination of transdermal uptake, added color, UV-protection, water resistance, favorable texture/smoothness, preservative/antibacterial action, and/or time-release of fragrance molecules. The terms “modified” and “modification” include but are not limited to: (a) the formation of new copolymers from the polyamines; (b) the impregnation of polyamines within, or attachment of polyamines to, porous inorganic or organic microbeads; (c) the formation of inorganic/organic hybrid materials from the polyamines; and/or (d) the attachment of inorganic and/or organic molecules to the polyamines to provide additional functionality (added odor absorbance, fragrance, antibacterial activity, preservative, coloration, added texture, or other useful functions).

Existing well-known odor absorbing agents may also be used in the final product (in minor amounts compared to the amount of modified polyamines present) to further enhance the overall performance, if so desired.

The odor absorbing compositions of the invention will be composed of preferably from about 1 to about 99% of odor absorbing ingredient(s), of which at least 50% of the total amount of odor absorbing ingredient(s) will be a modified polyamine(s). The odor-absorbing compositions will be composed of preferably from about 1 to about 99%, more preferably from about 2 to about 75%, and most preferably from about 5 to about 50% of modified polyamine(s). The compositions may include from 0 to about 50% of an additional inorganic oxide material or mixtures thereof (e.g., silica, titania, alumina, aluminosilicates, and the like), from 0 to about 10% of additional odor absorbing agents (cyclodextrins, carbonate salts, bicarbonate salts, zinc salts, aluminum salts, zeolites, ionic polymers, and the like), from 0 to about 1% of a fragrance enhancing agent, from 0 to about 1% of a preservative, from 0 to about 5% of a coloring agent, from 0 to about 50% of a surfactant, from 0 to about 90% of other ingredients known as additives in similar personal care products, and (if so desired) solvent(s) in appropriate quantities (water, alcohol, propylene glycol, etc.).

### DETAILED DESCRIPTION OF THE INVENTION

The terms “a” and “an”, as used herein and in the appended claims, mean “one or more” unless otherwise indicated herein.

In the present invention, polyamines (including polyalkylenimines) are refined and modified so that they become more suitable for use in personal care products. The large capacity of polyamines to absorb compounds responsible for body odor has been confirmed. In addition, polyamines are hydrolytically stable, thus leading to potential improved product stability and effectiveness. The modified polyamines of the invention are used as the main or major odor absorbing component in novel personal care products, including but not limited to underarm deodorant, foot odor-controlling agents, sunscreen, and hand and body lotions and wash/soap products. If so desired, these modified polyamines will provide formulations that have improved clarity when applied to the skin (hence leaving no visible residue) and a soft/smooth texture (hence providing a pleasant feel). Additional benefits may also be provided by modification of the polyamines, as described herein. The following sections will outline the present invention to combat human body odor utilizing modified polyamines, including descriptions of the modifications, additional ingredients to be included in product formulations, and methods to develop consumer-friendly formulations based on this class of potent odor absorbents. All component percentages, ratios, and parts herein are by weight, unless otherwise stated.

The products produced according to the present invention can be in the form of sticks (emulsion or suspensoid), creams, gels, powders, roll-on formulations, sprays, bars, and other forms of personal care products known in the art. The performance of the novel active ingredients, i.e., modified polyamines (or, multiple amine-containing polymers), derives from charge-charge complexation with the acids produced by bacteria present on human skin. When clusters of positive charges are copiously dispersed in a preparation, multiple electrostatic interactions take place between a given acid group and the clustered cations on the polymer backbone (or pendant branches), enhancing their binding affinity and hence sequestering all odor-producing molecules. Additional odor absorbing active ingredients known in the field of personal care products (aluminum salts, zinc salts, carbonate salts, bicarbonate salts, zeolites, cyclodextrins, ionic polymers, and the like) may also be included in the current invention in small quantities to supplement the action of the modified polyamine(s), if so desired. Additives known in the industry are optionally permitted for use in the current invention (i.e., solvents, rheology modifiers, surfactants, fragrances, preservatives, anti-bacterial agents, colorants, etc.).

For the purposes of this description, the terms “multiple amine-containing polymer”, “amine-containing polymer” and “polyamines”, as used herein and in the appended claims, all refer equally to polymers that contain amine groups either within or pendant from the polymer backbone.

For the purpose of this description, the term “amine group” describes primary, secondary, and/or tertiary amine groups. The polymer may also contain quaternary amine groups, but the inclusion of quaternary amine groups without primary, secondary, or tertiary amine groups is insufficient to qualify such a polymer as an “amine-containing polymer” as described herein.

A detailed description of the necessary and optional components of the present invention is provided below.

The amine-containing polymer may come from natural sources or from synthetic preparation. The amine-containing polymer may also optionally contain other reactive groups (i.e., non-amine); these other reactive groups may be but are not limited to hydroxyl, thiol, epichlorohydrin, carbonyl, halide, vinyl, allyl, and carboxylate. The amine-containing polymer may be a homopolymer or a copolymer. Examples of amine-containing polymers from natural sources include amine-containing polysaccharides and amine-containing polypeptides. Examples of synthetic amine-containing polymers include polyethylenimine (PEI) and PEI derivatives, poly(vinylamine), poly(diallylamine), poly(allylamine), copolymers of diallylamine and allylamine, and copolymers containing diallylamine and/or allylamine, and condensation polymers formed from polyamine monomers and monomers with two or more amine-reactive groups. Presently preferred embodiments of the invention include the synthetic polymers PEI and PEI derivatives, poly(vinylamine), and polymers containing diallylamine or allylamine. While not wishing to be bound by theory, it is believed that the ethylene segments of PEI exhibit strong affinity toward the alkyl moieties of the odor molecules. Such hydrophobic interactions synergistically enhance the electrostatic complexation of the positive-negative charges of the polyamine-acid pair. PEI can be derivatized with molecules containing such reactive groups as halohydrins, epoxides, organic acids, α,β-unsaturated organic acids, and carbonyls. PEI polymers and derivatized PEI polymers are commercially available from (for example) Nippon Shokubai and BASF.

Modification of these basic polymers to allow their use in personal care products is accomplished, according to the present invention, via one or more of the following methods: (a) reaction with dermatologically compatible aqueous-soluble or oil-soluble polymers to form novel bio-compatible copolymers; (b) the impregnation of polyamines within, or the attachment of polyamines to, porous inorganic and/or organic microbeads; (c) the formation of inorganic/organic hybrid materials from the polyamines; and/or (d) the attachment of inorganic and/or organic molecules to provide additional functionality (e.g., added odor absorbance, fragrance release, antibacterial action, preservation, coloration, added texture, increased affinity for skin and/or hair, and other functionalities as would be known to those of skill in the art of personal care products). The purpose of all such modifications is to facilitate the follow-on formulation effort, to reduce transdermal absorption (with its attendant systemic consequences) of the polyamine-based active ingredients, and to further enhance the overall product properties as described herein.

The polyamines can be reacted with carboxylic acid, anhydride-substituted, aldehyde-substituted, epoxide-substituted, or otherwise functionalized polysaccharides to form a copolymer. Other carboxylic acid, anhydride, aldehyde, epoxide, or other reactive group-containing polymers can also be reacted with the polyamines to form novel copolymers. All such reactive polymers (water- and oil-soluble) are to be considered part of the present invention. The oil-soluble polymers (e.g., siloxanes, lipids or oils) may be present as a fine emulsion, so that coupling occurs at the interface between the water and oil phases. Alternatively, the two reactants (polyamine and oil-soluble reactive polymer) can be mixed in a mutual solvent in order to form block or graft copolymers. For example, silicones, derivatized to contain carboxylic acids, carbonyls, epoxides, hydroxyls, or other reactive groups, can be joined with the polyamines to form block or graft copolymers. Such siloxane-containing copolymers provide excellent texture (softness) and permit gas permeability to allow for skin “breathing”.

Examples of polymers that may be coupled with the polyamines, as described herein, include the parent (where applicable), copolymers of, and any derivitized versions of: poly(ethylene glycol), poly(propylene glycol), poly(urethane), poly(ethylene), poly(acrylamide), poly(styrene), poly(vinylpyrrolidone), poly(methylmethacrylate), poly(acrylic acid), poly(aspartic acid), poly(dimethylsiloxane), poly(vinylalcohol), cellulose, methyl cellulose, hydroxyethyl cellulose, hydroxypropyl cellulose, dextran, agarose, poly(vinylphenol), poly(vinylacetate), poly(itaconic acid), poly(maleic acid), poly(maleic anhydride), poly(ester), nylon, and the like. To facilitate the coupling reactions, a catalyst may be required (i.e., acid, base, etc.).

The polyamines can also be linked to their partner polymer(s) by means of crosslinkers. The term “crosslinkers” as used herein describes molecules that contain two or more functional groups that form bonds with the reactive groups of the amine-containing polymer (amine and non-amine reactive groups) and with the polymer to be coupled with the polyamine. The crosslinkers can function to bind the amine-containing polymers together to form their own aggregate(s), or to attach the amine-containing polymers to a partner polymer(s) (in either soluble or aggregate form), or to impart the polyamines onto porous inorganic particles (as discussed more fully below). As a simple illustration, the amine groups of the amine-containing polymer are also the reactive groups used to effect attachment to another species (an organic polymer, an inorganic particle, etc.).

Polyamines that contain primary and/or secondary amines are particularly preferred in this embodiment. Those of skill in the art of chemistry will recognize that primary and secondary amines possess much greater versatility in bond formation than do tertiary amines, thereby broadening the types of potential crosslinkers that can be utilized. The reactive groups of the crosslinker should be present in sufficient quantity to effect attachment, but preferably in a sub-stoichiometric amount relative to the amine groups of the polymer. It is particularly desirable in this embodiment that the crosslinker reactivity with amine groups be significant, so that the cross-linkers react efficiently to bind the polymers together. It is also desirable, but not requisite, that the basicity of the nitrogen atoms that participate in the crosslinking reaction be substantially unchanged after the reaction. Specific amine-reactive groups include alkyl halides, isothiocyanates, isocyanates, acyl azides, N-hydroxysuccinimide esters, sulfonyl chlorides, aldehydes, glyoxals, epoxides, oxiranes, carbonates, arylating agents, imidoesters, carbodiimides, anhydrides, and halohydrins. In a presently preferred embodiment, the cross-linker contains halohydrin or epoxide reactive groups. Examples of these include, but are not limited to, 1,3-dichloro-2-propanol (Sigma Aldrich Corporation), 1,4-butanediol diglycidyl ether (Resolution Performance Products), and low molecular weight epoxypropoxypropyl terminated polydimethylsiloxanes (Gelest, Inc.).

Specific control over the polyamine nanostructure can be achieved via the use of appropriate crosslinking molecules. For example, use of an appropriate amount of a difunctional crosslinking molecule will provide (on average) polymers with a “dumbbell” shape (two polymer moieties bridged by an organic linker). Further reactions with crosslinking molecules can be easily envisioned to create extended one-, two-, and three-dimensional polymeric nanostructures, depending upon: (a) the type of crosslinker(s) used (difunctional, trifunctional, etc.); (b) the number of different types of crosslinkers used; and (c) the amount of each crosslinking molecule used. The newly formed nanostructured polyamines can then be coupled with other functional polymers to create new copolymers with appropriate functionality. Indeed, we have observed enhanced performance (for example) in the area of water-resistance and substrate binding strength when such nanoarchitectural control (the ability to prepare modified polyamines with “tunable” nanostructure) is utilized in the generation of copolymers appropriate for use in personal care products, relative to polymers without such nanostructural modification(s).

In another embodiment, the amine-containing polymer also contains non-amine reactive groups. The presence of non-amine reactive groups is particularly valuable when the amine groups of the polymer are exclusively or almost exclusively tertiary amine groups. Examples of non-amine reactive groups that are of use in the present invention include hydroxyls, thiols, and carboxylic acids. In a presently preferred embodiment, the reactive groups are hydroxyls. It is desirable that the crosslinking reaction does not affect the basicity of the amines in the resulting conjugate. The crosslinking molecule must contain two or more functional groups that can react with the non-amine reactive group(s) of the amine-containing polymer. A catalyst may optionally be included to facilitate crosslinking (i.e., acid or base, etc.). Hydroxyl-reactive functional groups that can be used include epoxides, halohydrins, oxiranes, carbonyl diimidazole, N, N′-disuccinimidyl carbonate or N-hydroxysuccinimidyl chloroformate, alkyl halogens, isocyanates, and N-methylol ureas. Thiol groups react with haloacetyl and alkyl halide derivatives, maleimides, aziridines, acryloyl derivatives, arylating agents, and thiol-disulfide exchange reagents such as pyridyl disulfides, disulfide reductants, and 5-thio-2 nitrobenzoic acid. Carboxylate-reactive groups include diazoalkanes and diazoacetyl compounds, carbonyl diimidazole, carbodiimides, and N-methylol ureas. Preferred crosslinkers here are diepoxides (Resolution Preformance Products) and N-methylol ureas such as dimethyloldihydroxyethyleneurea (PatCoRez P-53, BF Goodrich). These crosslinkers are useful when the polyamines are to be attached to polysaccharide gels, agars, dextrans, cellulosics/cellulosic derivatives, glycerol, polyethylene glycol or polypropylene glycol chains, polyvinylalcohol, poly(hydroxyethylmethacrylate), poly(hydroxy ethylacrylate) and other hydroxy-containing polymers/copolymers. In addition, protein and protein digests can be coupled with the polyamines to ease follow-on formulation work. The odor absorbing actives can be combined with collagen, either chemically or physically.

In yet another approach to utilize the polyamine-based active ingredients (in an unmodified or otherwise modified state), polyamines are placed into intimate contact with a microbead. By “microbeads” is meant relatively spherical particles on the order of about 1 to 100 microns in size that may or may not be porous in nature. By “intimate contact” is meant that the polyamines are placed in or on the microbeads; that is, they can be adsorbed or absorbed on, chemically attached to, or physically entrapped in the microbeads. Example particle matrices include both inorganic (such as silica, titania, zinc oxide, etc.) and organic (such as crosslinked vinyl, styryl, acrylate, and methacrylate polymers) varieties.

The microbeads can be formed in the presence of the polyamines by sol-gel processes (for inorganic beads) or free-radical polymerizations (for organic beads) by methods know to those skilled in the art. Other approaches can be exploited such as diffusion/impregnation of polyamine-containing solutions into the beads (if porous), followed by drying. Various crosslinkers can also be used in this approach to effect a covalent attachment of the polyamine to the microbeads (i.e., surface and/or pore coating). In addition to the crosslinkers described above for attachment of organic moieties, functional silanes can be used to provide attachment to inorganic microbeads. For example, epoxide-containing triethoxysilanes are readily available (Gelest, Inc.) and can be used to attach an organic polymer to an inorganic microbead. The silane is attached to the inorganic particle via sol-gel methods and the polyamine is subsequently reacted with the newly functionalized microbead. Conversely, the polyamine can be reacted with the silane to provide a sol-gel reactive polyamine that can be incorporated into an inorganic microbead via sol-gel methods. Additionally, trialkoxysilane-functionalized polyethylenimines are commercially available (Gelest, Inc.) and may be used in the current embodiment. Many alternatives for incorporation of a polyamine into a microbead can be envisioned. All those reasonably expected of one who is skilled in the art of polymer science and technology, chromatography or ion exchange shall fall within the scope of this invention.

The term “inorganic/organic hybrid material” as used herein refers to a nanocomposite material comprising an organic component that is covalently bound to an inorganic component (including a mircobead), preferably an inorganic oxide or hydroxide (e.g., SiO₂, TiO₂, ZnO, Al₂0₃, Mg(OH)₂, etc.). The inorganic components or resulting inorganic/organic hybrid materials preferably have amorphous structures, high surface areas, and large pore volumes. Alternatively, the inorganic oxides can be nanocrystalline materials (of sizes from about 1 nm to 1000 nm). For example, many nanocrystalline oxide (and related) materials (MgO, Mg(OH)₂, Al₂0₃, etc.) with high surface areas are known (see “Nanoscale Materials in Chemistry”, K. J. Klabunde (Ed.), Wiley, New York, 2001). Another embodiment allows for the use of oxide nanoparticles, described in U.S. Pat. No. 5,795,565 (L'Oreal) as effective sunscreen actives. The disclosure of U.S. Pat. No. 5,795,565 is incorporated herein by reference. Such oxide nanoparticles are commercially available from a number of sources (see, U.S. Pat. No. 5,795,565 disclosure).

The inclusion of inorganic components (including microbeads) may offer several beneficial effects: (a) providing increased resistance to product removal by washing away using aqueous media (including but not limited to sweat, water, and saline solutions); (b) providing increased biocompatibility of the polyamine by reducing skin sensitivity; (c) preventing transdermal uptake of the polyamine; (d) providing protection from harmful UV radiation, upon use of known sunscreen active inorganic components (TiO₂, ZnO, etc.); (e) providing desirable coloration via use of certain inorganic pigments (Fe₂O₃, Cr₂O₃, etc.).

The formation of the inorganic/organic hybrid composite materials can be accomplished via standard sol-gel chemistry (see “Sol-Gel Science”, C. J. Brinker and G. W. Scherer, Academic Press, Boston, 1990) utilizing alkoxide-containing silanes that have an amine-reactive functional group (e.g., epoxide, etc.) in a manner similar to that described above for attachment of polyamines to inorganic microbeads. For example, triethoxysilanes containing halides and epoxides are known, and can be utilized to form the inorganic/organic linkage by: (a) formation of an inorganic “sol” (after partial hydrolysis of the necessary components) containing these reactive silanes (now integrated within a growing inorganic oxide material) followed by subsequent reaction with a polyamine to form covalent linkages between the inorganic “sol” and the organic polyamine, (b) derivitization of the polyamine using such silanes prior to any sol-gel process, with subsequent addition of the necessary inorganic sol-gel precursors to form the final hybrid material, or (c) reaction of a silane-derivatized polyamine with a pre-existing inorganic material (amorphous, nanocrystalline, etc.). Further, the polyethylenimine polymers that contain silicon alkoxide groups can be utilized here to create hybrid materials via sol-gel processes in the presence of inorganic alkoxides (i.e., tetraethylorthosilicate, titanium isopropoxide, aluminum isopropoxide, and the like) and pre-existing inorganic materials under hydrolytic conditions.

In a further embodiment of the current invention, high surface area and mesostructured oxide materials can be used as the inorganic component to which the polyamine is attached. The mesostructured inorganic materials are formed via use of non-reactive organic polymers acting as “templates” during a sol-gel preparation of the material. For example, Stucky et. al. (Science, 1998, 279, 548) reported formation of mesoporous oxide materials (SBA materials) via use of commercially available ethylene oxide-propylene oxide block copolymers (Pluronics™, from BASF). Other such organic templates are also known to form ordered porous inorganic materials. For example, trialkylammonium salts have been utilized as templates for the generation of high surface area mesoporous oxide materials (MCM materials, from Mobil Corporation, Beck et al. J. Am. Chem. Soc., 1992, 114, 10834). Use of the organic templates may be either concurrent with the generation of the inorganic/organic hybrid material or prior to said hybrid formation. Hence, attachment of the polyamine to the mesostructured material can take place after formation of said material or during its formation (in a sol-gel process). In all cases where an organic template is used, the template can be easily removed via a simple extraction process leaving the mesostructured inorganic/organic polyamine-containing hybrid material.

Another major advantage of polyamine actives is their compatibility with a new generation of antimicrobials, which, in contrast to traditional compounds, exhibit low toxicity to humans. Various short (fifty amino acids or less) cytotoxic polypeptides have been identified (Malony, et al., Biopolymers (Peptide Science) 37: 105-122 (1995)). They share the common trait of a high content of arginine and lysine residues, and carry a net positive charge at physiological pH. The mechanism of toxicity appears to be cell lysis, mediated by electrostatic coordination of the peptide to the cell wall.

U.S. Pat. No. 5,300,287, issued to Park, teaches the derivatization of polyethylenimine, particularly with polyethylene glycols, to form graft polymers that exhibit antimicrobial and antifungal activity. These polymers are particularly directed towards use in ophthalmic products and contact lens care solutions. Such polyethylene glycol/polyethylenimine copolymers can be used in the current embodiment (subsequent to modification as described herein), thus providing inherent antimicrobial activity. U.S. Pat. No. 6,034,129, issued to Mandeville et al, teaches the use of cationic polymers to treat bacterial infections in mammals, specifically humans. The polymers described in the patent have amino or ammonium groups pendant from the polymer backbone.

Attachment of other inorganic and/or organic molecules to the polyamine(s) is another modification that can provide additional functionality. Reactive organic dyes can be covalently attached to the polyamines to provide coloration. The quantity and type of dye can be varied such that a wide range of colors are available. Examples of dyes that are commercially available and reactive toward amines include those containing cyanuric chloride or vinyl sulfone functional groups. Molecules known as fragrance enhancing agents can be attached using linkages that are hydrolysable. Thus, the fragrance molecules can be slowly released over time (the rate of release can be tuned via the degree of crosslinking, strength of the hydrolysable linkage, etc.). Molecules that are known as preservatives (i.e., radical scavenger, antimicrobial, antifungal, etc.) can also be attached to the polyamine(s) to impart such preservation action to the polyamine. Small molecule siloxanes or fluorocarbons can be attached to alter the hydrophobicity of the polyamine(s). Such siloxane and fluorocarbon modifications provide enhanced water resistance and improved textural feel. Inorganic odor absorbing actives may be attached directly to the polyamine(s) utilizing coordination chemistry involving the amine groups and metal centers. For example, amines are known to coordinate to and solubilize certain inorganic salts, many of which are known odor absorbing ingredients (i.e., zinc, aluminum, and magnesium ions). As long as the metal centers are not coordinatively saturated (irreversibly) with amine groups from the polyamine(s), they will maintain the ability to bind odorous molecules (amines, mercaptans, sulfides, etc.), hence continuing the deodorizing activity. Along related lines, organic molecules that are known as odor absorbing actives (i.e., cyclodextrins, etc.) can be covalently attached to the polyamine(s) to further enhance the odor absorbing action of the polyamine(s).

In developing a fully formulated personal care preparation, the present invention permits the use of other ingredients that are not attached to the modified polyamine(s). Note that these potential adjunct materials must all be compatible with the odor-absorbing polyamine(s), making formulation a straightforward task. These optional ingredients may be fragrances, preservatives, additional odor absorbing agents, additional polymers, colorants, antiperspirants, rheology modifiers, moisturizing agents, vitamins, solvent(s), etc. These components either reside in an aqueous phase, an oil phase, or are loosely associated with the modified polyamine(s), but they are not chemically bound to the polyamine(s).

The odor absorbing personal care preparation optionally includes the use of fragrances in amounts that are non-irritating to the average user's skin and/or respiratory system, but that can be detected by the user's sense of smell prior to and after appropriate use of a product. Typically, amounts of added fragrance would preferably not exceed 1% of the final composition. Suitable fragrances may be chosen from those known to one skilled in the art.

The personal care preparation of the invention optionally allows for the use of preservatives. Preferred preservatives are water soluble and are effective on both bacteria and fungi (so called broad spectrum preservatives). A limited spectrum preservative such as one that is only effective on a single group of microorganisms can be used in combination with a broad spectrum preservative or with other limited spectrum preservatives with complimentary and/or supplementary activity. A mixture of broad spectrum preservatives can also be used. Many preservatives suitable for use are described in U.S. Pat. No. 5,534,165, issued to Pilosof, which is incorporated herein by reference. Typically, the total amount of added preservative would preferably not exceed 1% of the final composition.

The odor absorbing personal care preparation optionally allows for the use of adjunct odor absorbing components. Examples of suitable odor absorbing agents include, but are not limited to, zinc salts, magnesium salts, aluminum salts, carbonate salts, bicarbonate salts, cyclodextrins, ionic polymers, cationic polymers, anionic polymers, zeolites, silica gel, silica molecular sieves, activated alumina, kieselguhr, fullers earth, montmorillonite, smectite, attapulgite, bentonite, polygorskite, kaolinite, illite, halloysite, hectorite, beidellite, nontronite, saponite, hormite, vermiculite, sepiolite, chlorophyll, soda lime, calcium oxide, chitin, potassium permanganate, and activated carbon, and mixtures thereof. Typically, cumulative amounts of these added odor absorbing components would not exceed 10% of the final composition.

The current embodiment optionally allows color enhancement by the addition of dyes (organic, not attached to the polyamine) and pigments (inorganic in nature) to provide coloration to the final product, if so desired.

The current embodiment optionally allows use of surfactants to provide reduced surface tension and allow improved spreading of the product on the human skin. The surfactants used must be compatible with the modified polyamine(s) as described herein. Nonlimiting examples of suitable surfactants include polyalkylene oxide polysiloxanes, copolymers of siloxanes and ethylene oxide, copolymers of ethylene oxide and propylene oxide, emulsifying surfactants having an HLB (hydrophobic lipophilic balance) value below about 12, and emulsifying surfactants having an HLB value of about 12 or above, and mixture(s) thereof.

The current embodiment optionally allows the use of other beneficial ingredients known in the art of skin protection, moisturizing, and cleansing. The components are typically oil-soluble and can be used in conjunction with the modified polyamine(s) without interference. For examples, an oil phase may contain skin protectants (such as vitamin A, cod liver oil, cocoa butter, shark liver oil, dimethicone, petrolatum, mineral oil, jojoba oil and lanolin) and/or emollients (such as tocopherol, tocopherol acetate, triglycerides, vegetable oils, and mineral oils). The microstructure formed between the oil phase and the active-containing phase (which may be aqueous or almost dry) is made stable by the judicious use of surfactants or emulsifiers, as known in the art.

Alcohols are permitted for use as solvents and/or an antiseptic ingredient. For example, ethanol and 2-propanol are known to act as antiseptic agents. The current embodiment preferably has from 0 to about 90%, more preferably from 0 to about 75%, and most preferably from 0 to about 50% alcohol. Both ethanol and 2-propanol (including mixtures thereof) are preferred in the current embodiment.

The fully formulated personal care preparations may have varying consistency depending on their intended use. The preparations may be used as a stick (emulsion or suspensoid), a gel, a lotion, a cream, a skin patch, a powder, a roll-on or a spray-on product. Various agents can be used to modify the rheology of the composition. Examples of such components include, but are not limited to, gel-forming inorganic oxides (bentonite, silica, etc.) and organic polymers (alginate, xanthan gum, guar gum, tragacanth gum, starch, cellulose and modified celluloses, polyvinylpyrrolidone, polyvinylalcohol, polyvinylacetate, polyacrylic acid, etc.). The thickener or other component should be compatible with the modified polyamines, such that the ability of the modified polyamines to absorb odors is not diminished, nor is the ability of the component to provide the desired consistency. It is preferred that the thickening component(s) does not complex with the modified polyamines.

The methods and chemistry described herein can be used to produce products for use as standard deodorants (stick, roll-on, etc.), skin cleansers/moisturizers with deodorizing action, hair shampoos/conditioners with deodorizing ability, artificial tanning and/or sunscreens with deodorizing action, foot odor controlling products, and any number of other products related to the personal care industry.

### EXAMPLES

### Example 1

An amine-containing polymer or oligomer, such as poly(ethylenimine), poly(allylamine hydrochloride), or poly(lysine), is reacted with one or more polymer(s) containing amine reactive groups (e.g., epoxide, halohydrin, etc.) to form a copolymer (the modified polyamine) with properties representative of the individual polymeric components. Therefore, depending upon the components of the modified polyamine, various desirable properties can be achieved. For example, when an epoxide-containing polydimethylsiloxane is coupled with poly(ethylenimine) using different PEI-to-siloxane ratios, varying (and tunable) degrees of water and/or detergent resistance (up to and including complete resistance) and softness/smoothness are imparted into odor absorbing films cast using the new modified polyamines. In addition, skin irritation of the siloxane-modified polyamine(s) is greatly reduced or eliminated versus that of PEI alone.

Various crosslinkers can be employed to modify polyamines and/or further enhance the properties of the already-modified polyamines (described above). For example, crosslinking individual PEI polymers using an epoxide-terminated polydimethylsiloxane can provide extended networks which will allow for increased durability of the product when applied to a substrate. The polydimethylsiloxane linkers allow for added flexibility to provide a smooth and pleasant feel. In addition, the polydimethylsiloxanes allow for oxygen permeability, hence allowing the skin to “breathe”. Careful control over the type and amount of a given crosslinker can lead to formation of polymeric nanoarchitectures with (on average) a well-defined structure. For example, PEI (known to be a branched polymer that is somewhat compacted) can be bridged using difunctional cross-linkers to form dumbbell-type structures. Further, use of multi-functional crosslinkers can provide more complicated nanostructures (extended linear structures, three-pronged moieties, highly-branched dendritic structures, nets, 3-dimensional networks, etc.). Many examples of organic molecules with multiple amine-reactive functional groups are known (e.g., trimethylolpropane triglycidylether, triglycidyl isocyanurate, triphenylolmethane triglycidylether, etc.). In addition, commercially available polymers that have been functionalized with small amounts of amine-reactive groups are appropriate crosslinkers in the current embodiment (e.g., polyethylene-co-glycidylmethacrylate (Aldrich), poly(epoxycyclohexylethylmethylsiloxane-co-dimethylsiloxane) (Gelest), and other functionalized polymers).

Other polymeric components can be reacted with the PEI portion of the modified polyamine to impart additional properties into the copolymer, while maintaining the beneficial properties of the other two components. For example, polymers that are known to provide improved film-forming capabilities can be introduced.

The new modified polyamines described herein can be delivered to the substrate (e.g., the skin) as solutions (aqueous, alcohol, or a mixture of the two) or emulsions (aqueous, alcohol, or a mixture), depending on the solubility or lack thereof of the copolymers. The final formulation can be in the form of a stick (suspensoid or emulsion), spray, lotion, cream, etc. This and all other formulations and solutions mentioned in this document may additionally contain other odor absorbing components, fragrances, wetting agents, opacifiers, thickeners, defoamers, surfactants (anionic, cationic, nonionic, amphoteric, zwitterionic, or mixtures thereof), sequestering agents, emollients, medicines (drugs), vitamins, dispersing agents, conditioners, alcohols, oxidizing agents, antioxidants, reducing agents, antibacterial agents, preserving agents, and the like, as well as mixtures thereof.

### Example 2

Another modification of an amine-containing polymer or oligomer, such as poly(ethylenimine), poly(allylamine hydrochloride), or poly(lysine), constitutes the reaction of functional molecules to form covalent linkages between the polyamine and said functional molecules. Thus, modified polyamines are provided with added functionality. For example, one or more of the same or different dye molecules can be covalently bonded, by methods known in the art, to the polyamine(s), to provide modified polyamines that are colored and still retain their odor absorbing properties. Additional functional molecules can also be covalently attached to the modified polyamines to provide multiple (cumulative) benefits, as described previously. Optional ingredients (as listed in example 1 and throughout this document) can also be added to provide an appropriate formulation for use on a substrate (i.e., skin).

### Example 3

Functional molecules can be covalently bonded, by methods known in the art, to the polyamine part of the modified polyamines (synthesized as described in example 1), to provide modified polyamines with additional properties. For example, one or more of the same or different dye molecules can be covalently bonded, by methods known in the art, to the polyamine part of the siloxane-modified polyamines (as described in example 1), to provide colored, odor absorbing films with tunable water/detergent resistance, enhanced texture, etc, when such films are cast using these modified polyamine(s). Optional ingredients (as listed in example 1 and throughout this document) can also be added to provide an appropriate formulation for use on a substrate (i.e., skin).

### Example 4

An amine-containing polymer (either the parent polyamine or a modified version thereof, as described in examples 1-3), is reacted with, by methods known in the arts of chemistry and materials science, (for example) an epoxide-containing alkoxysilane, forming a silicon alkoxide-containing polymer. This alkoxide-functionalized polyamine is then coupled to an inorganic oxide material (amorphous, nanocrystalline, mesoporous, microbead, and the like) via a sol-gel type reaction forming a hybrid inorganic/organic nanocomposite odor absorbing material. Examples of permitted oxide types include, but are not limited to, silica (available from Degussa Huls, Cabot, Waker-Chemie, etc.), titanium dioxide (available from Degussa Huls, Warner Jenkinson Cosmetic Colors, Kerr-McGee, Hilton Davis, Engelhard Corp., etc.), and zinc oxide (US Cosmetics, Whittaker, Clark & Daniels, Nanophase, etc.). The mode of coupling proceeds via the alkoxide groups being hydrolyzed under acidic or basic conditions, and the silanol groups subsequently formed reacting with surface hydroxyls of the inorganic component to form stable covalent linkages, upon elimination of water. Heat may or may not be required in order for the coupling reaction to occur. The quantity of silane used in the reaction is variable, but typically would constitute only a small fraction of the overall reagent quantities. For example, in theory only one silane per polymer chain is required to provide inorganic/organic coupling. Because of statistical considerations, an amount greater than one silane per polymer chain should be used, but this amount should be the minimum amount necessary to yield efficient coupling, which amount can be determined without undue experimentation. The organic components of the new hybrid material will reside on the surface; hence, high surface area and highly porous inorganic oxides are preferred (but not necessary) starting materials to provide more organic incorporation. The new hybrid material exhibits various properties depending upon the amount and type of organic component(s) used. In addition, the inorganic component will provide added benefits, nonlimiting examples of which are: improved durability, elimination of transdermal uptake, added odor absorbing effects, UV protection (i.e., using TiO₂, ZnO, etc.), and coloration (if a colored pigment-type material is used). The inorganic/organic hybrid material can be used in powdered (dried) or gel (wet) form. Modified polyamines (as described in examples 1-3) may also be included in a final formulation if so desired. Optional ingredients (as listed in example 1 and throughout this document) can also be added to provide an appropriate formulation for use on a substrate (i.e., skin).

### Example 5

As an alternate method to prepare hybrid inorganic/organic nanocomposite materials, an inorganic oxide material (amorphous, nanocrystalline, mesoporous, microbead, etc.) is functionalized using an epoxide-containing alkoxysilane via sol-gel type reactions, providing an epoxide-functionalized material. This amine-reactive inorganic material is then reacted with an amine-containing polymer (either the parent polyamine or a modified version thereof, as described in examples 1-3). Reaction occurs at the epoxide sites distributed throughout the surface of the inorganic material. Hence, a hybrid inorganic/organic material is formed with the organic components covalently bound to the surface of the inorganic component. The degree of surface coverage is determined by the amount of polyamine used. High surface area and highly porous inorganic materials will permit the maximum incorporation (by weight %) of polyamine(s). The inorganic/organic hybrid material can be used in powdered (dried) or gel (wet) form. Optional ingredients (as listed in example 1 and throughout this document) can also be added to provide an appropriate formulation for use on a substrate (i.e., skin).

### Example 6

As a further method to prepare hybrid inorganic/organic nanocomposite materials containing polyamines and modified polyamines (as described above in examples 1-3), a polyamine is reacted with an amine-reactive alkoxysilane. The alkoxysilane-containing polyamine thus formed is subsequently used in a sol-gel reaction whereby an alkoxide compound (e.g., titanium isopropoxide, silicon ethoxide, aluminum isopropoxide, etc.) is polymerized to form an inorganic oxide gel. Therefore, the polyamine is directly incorporated into the growing inorganic network. This method of hybrid inorganic/organic material formation provides a material with organic components throughout the entire sample (i.e., on the surface and in the bulk). Hence, larger amounts of polyamine are easily incorporated, if so desired. The inorganic/organic hybrid material can be used in powdered (dried) or gel (wet) form. Optional ingredients (as listed in example 1 and throughout this document) can also be added to provide an appropriate formulation for use on a substrate (i.e., skin).

### Example 7

Another refinement on the preparation of inorganic/organic hybrid materials containing polyamine(s) is provided by following example 6, but including a non-reactive organic template into the sol-gel reaction mixture to provide a high surface area material with an ordered mesostructure. The organic template (polyalkylene oxide block copolymers, trialkylammonium salts, etc.) can be used in quantities according to known procedures, to effect the formation of pores with a known size distribution. The organic template is easily removed via extraction using an organic solvent, alcohol, and/or water. The resulting mesostructured hybrid material can then be used as an effective odor absorbing material, with the benefits described above and the added benefit of having large pores (mesopores) that easily allow organic odor molecules to penetrate into the material for effective absorption by the polyamine components. Although preferred, the mesoporous hybrid material formed does not necessarily need to have a well-ordered pore structure (i.e., a disordered or worm-hole mesostructure is permitted). The inorganic/organic hybrid material can be used in powdered (dried) or gel (wet) form. Optional ingredients (as listed in example 1 and throughout this document) can also be added to provide an appropriate formulation for use on a substrate (i.e., skin).

1. A modified polyamine which comprises a hybrid inorganic/organic material comprising a polyamine and an inorganic material having one or more characteristics selected from the group consisting of amorphous structures, high surface areas, large pore volumes, and nanocrystalline structures.

2. A modified polyamine according to claim 1 wherein the inorganic material is an inorganic oxide material.

3. A modified polyamine according to claim 1 wherein the polyamine is selected from the group consisting of amine-containing polysaccharides, amine-containing polypeptides, polyethylenimine, polyethylenimine derivatives, poly(vinylamine), poly(diallylamine), poly(allylamine), copolymers of diallylamine and allylamine, copolymers containing diallylamine or allylamine, copolymers containing diallylamine and allylamine, and condensation polymers formed from polyamine monomers and monomers with two or more amine-reactive groups.

4. A modified polyamine according to claim 1 wherein the polyamine is selected from the group consisting of poly(lysine), polyethylenimine, polyethylenimine derivatives, poly(vinylamine), polymers containing diallylamine, and polymers containing allylamine.

5. A modified polyamine which comprises a polyamine impregnated into or attached to a porous inorganic or organic microbead.

6. A modified polyamine according to claim 5 wherein the polyamine is selected from the group consisting of amine-containing polysaccharides, amine-containing polypeptides, polyethylenimine, polyethylenimine derivatives, poly(vinylamine), poly(diallylamine), poly(allylamine), copolymers of diallylamine and allylamine, copolymers containing diallylamine or allylamine, copolymers containing diallylamine and allylamine, and condensation polymers formed from polyamine monomers and monomers with two or more amine-reactive groups.

7. A modified polyamine according to claim 5 wherein the polyamine is selected from the group consisting of poly(lysine), polyethylenimine, polyethylenimine derivatives, poly(vinylamine), polymers containing diallylamine, and polymers containing allylamine.

8. A modified polyamine which comprises a polyamine having inorganic molecules or organic molecules, or both, chemically attached to it.

9. A modified polyamine according to claim 8 wherein the polyamine is selected from the group consisting of amine-containing polysaccharides, amine-containing polypeptides, polyethylenimine, polyethylenimine derivatives, poly(vinylamine), poly(diallylamine), poly(allylamine), copolymers of diallylamine and allylamine, copolymers containing diallylamine or allylamine, copolymers containing diallylamine and allylamine, and condensation polymers formed from polyamine monomers and monomers with two or more amine-reactive groups.

10. A modified polyamine according to claim 8 wherein the polyamine is selected from the group consisting of poly(lysine), polyethylenimine, polyethylenimine derivatives, poly(vinylamine), polymers containing diallylamine, and polymers containing allylamine.

11. A modified polyamine which comprises a bio-compatible copolymer of a polyamine and a dermatologically compatible aqueous-soluble and/or oil-soluble polymer.

12. A modified polyamine according to claim 11 wherein the polyamine is selected from the group consisting of amine-containing polysaccharides, amine-containing polypeptides, polyethylenimine, polyethylenimine derivatives, poly(vinylamine), poly(diallylamine), poly(allylamine), copolymers of diallylamine and allylamine, copolymers containing diallylamine or allylamine, copolymers containing diallylamine and allylamine, and condensation polymers formed from polyamine monomers and monomers with two or more amine-reactive groups.

13. A modified polyamine according to claim 11 wherein the polyamine is selected from the group consisting of polyethylenimine, polyethylenimine derivatives, poly(vinylamine), polymers containing diallylamine, and polymers containing allylamine.

14. A nanostructured polyamine which comprises a polyamine reacted with one or more crosslinkers.

15-20. (canceled)
